(19) United States
(12) Patent Application Publication
(10) Pub. No.: US 2026/0334866 A1
(43) Pub. Date: Oct. 29, 2026

(54) System and Method for Delivering Non-Harmful Pain Stimuli and Evaluating Patient Response During Medical Examinations

(71) Applicant: B9 Stimulus Technologies, LLC, Oakland, MD (US)

(72) Inventors: Kyle Cooper, Littleton, CO (US); Geoffrey Miller, Elkridge, MD (US); Dennis Ngo, Springfield, VA (US); Vaishnavi Pinni, Ballwin, MO (US); Natasha Porwal, Sewickley, PA (US); Adam Schiavi, Middle River, MD (US); Iralde Sicilia, Sammamish, WA (US)

(21) Appl. No.: 19/662,210

(22) Filed: Apr. 29, 2026

Related U.S. Application Data
(60) Provisional application No. 63/796,536, filed on Apr. 29, 2025.

(51) Int. Cl. A61B 5/00

(52) U.S. Cl. CPC A61B 5/4824; A61B 5/6825; A61B 2560/0431

(57) ABSTRACT

A self-contained, portable, non-invasive thermal stimulation system including a handheld medical device for non-harmful thermal stimulus application is disclosed. Methods for application of the system are provided that utilize the thermal grill illusion to deliver a quantified, non-harmful pain stimulus to a patient in order to assess the patient’s response to such stimuli, thus allowing healthcare providers to apply the non-harmful pain stimulus in a quantified, standardized fashion without causing tissue damage. Methods for using the system to assess a patient’s consciousness, to perform pain research, evaluate pain disorders in different regions of the human body, and for longitudinal tracking of pain responsiveness combined with integration across multiple physiological monitoring modalities are also presented.

### PRIORITY TO RELATED APPLICATIONS

This application is a U.S. Patent Application which claims the benefit of U.S. Provisional Patent Application Serial No. 63/796,536 filed on Apr. 29, 2025, which is incorporated herein by reference.

### FIELD OF THE INVENTION

The present invention relates generally to human medical devices. Specifically, the present invention relates to a system and a method for assessing a patient’s response to pain stimuli applied to the patient by a portable non-invasive device. More specifically, the portable non-invasive device of the present invention is adapted to deliver standardized, non-harmful pain stimuli to a patient during medical examinations to evaluate a patient’s neurological function and consciousness.

### BACKGROUND OF THE INVENTION

Healthcare providers across a wide range of clinical settings by way of example, neurocritical care, emergency care, and general medical environments, routinely apply noxious or pain stimuli to patients to evaluate their physical responses. These stimuli are used to gather information about a patient’s condition, including, but not limited to, levels of consciousness, reflexes, and neurological function. The current standard of care relies on manual mechanical techniques such as fingernail or toenail bed pressure, arm or thigh pinching, or using a sternal rub, trapezius squeeze, or earlobe stimulation. These examinations are performed frequently—often hourly in intensive care unit (ICU) settings—and guide critical decisions such as treatment planning, medication adjustments, and the continuation of life support.

The existing approaches to pain stimulus assessment present two fundamental categories of problems. The first is direct patient harm. Repeated application of manual mechanical stimuli causes skin damage, bruising, abrasions, and subcutaneous hematomas. Vascular damage can accumulate from repeated trauma to the same areas. Beyond the physical injury, these techniques cause significant emotional distress—not only to patients but also to family members who witness visible harm being inflicted on their loved ones during what are already extremely difficult circumstances. The risk of infection from cumulative skin breakdown further compounds the problem, and in some cases these iatrogenic complications can extend ICU stays.

The second fundamental problem category is the lack of standardization. The intensity of pain stimuli used in examinations is not standardized, and the applied pain intensity may vary significantly among different healthcare providers and clinicians and across repeated examinations. Each individual clinician may apply a different amount of force, for a different duration, at slightly different locations on a patient in accordance with that clinician’s individual unique personal practice techniques. This provider-dependent variability leads to inconsistent assessments, making it difficult to reliably track a patient's neurological status over time. The inconsistency may further result in redundant imaging, unnecessary interventions, and longer hospitalizations.

Attempts to improve the process have met with limited success. For example, Fox et al., in U.S. Patent Application Publication No. US 2024/0306984 published on Sep. 19, 2024, discloses devices which apply standardized central and peripheral stimuli for assessments of stimulus response using electrical, laser, and mechanical processes for the stimulation. While improved accuracy and more consistent stimuli application are achieved, the devices disclosed by Fox et al. can still pose a risk for skin and underlying tissue damage to a patient if used improperly. The fundamental limitation of Fox et al., which is representative of current practice and the prior art, is that the stimulus modality itself—whether electrical, laser, or mechanical—carries inherent risk of tissue injury, particularly with the repeated applications required in clinical monitoring.

In view of the foregoing, it will be apparent to those skilled in the art from this disclosure that a clear and unmet need exists in the art for a device and application method to generate a pain stimulus and evaluate patient responses thereto without causing skin damage or injury to underlying tissue and which delivers a stimulus that is safe and completely non-injurious at any user-selected intensity setting, standardized and quantifiable so that results are comparable across clinicians and over time, adjustable to enable escalation protocols for graded assessments, and portable enough for bedside use across diverse clinical settings including ICUs, emergency departments, operating rooms, and pre-hospital environments. The apparatus and methodology of the present invention address these needs in the art, as well as other related clinical needs, all of which will become apparent to those skilled in the art from the accompanying disclosure.

### SUMMARY OF THE INVENTION

In order to address the aforementioned needs in the art, a novel medical device or system and methods for its use are provided that leverages the thermal grill illusion to provide a quantified, non-harmful pain stimulus to the patient. The thermal grill illusion is a sensory phenomenon well known in neuroscience in which warm and cold surfaces applied to the skin simultaneously in an interlacing pattern create a disproportionate painful burning sensation. Critically, the individual temperatures involved are not hot or cold enough to cause any tissue damage. The pain is entirely perceptual—a product of how the brain processes conflicting thermal signals—rather than the result of actual harmful stimulus. This will allow healthcare providers to apply the pain stimulus in a quantified, standardized fashion, without causing tissue damage.

In an embodiment, the medical device includes a housing or casing that houses the electronics which operate and control the device, a patient interface or thermal grill, an array comprised of a plurality of miniature Peltier elements adapted to heat and cool the thermal grill, a thermal control system, an active cooling system adapted to dissipate heat from a hot side of each of the plurality of Peltier elements, and a central processing unit or microcontroller with thermistor feedback adapted to receive inputs from a user interface system whereby a preselected temperature at the thermal grill is maintained.

In another embodiment, the casing that houses the electronics is in the form of an ergonomic, portable handheld unit adapted for one-handed operation by a healthcare provider at a point of patient treatment.

In an embodiment, the system is configured to integrate thermal stimulation, precision temperature control, and active cooling into a single ergonomic handheld unit.

In still another embodiment, the patient interface or thermal grill includes at least one pair of metal shields formed into a specific geometry or thermal grill structure whereby they interlock over the array of miniature Peltier modules.

In yet another embodiment, each of the metal shields is set to a specific preselected temperature which is regulated by the central processing unit.

In an embodiment, each of the metal shields is formed of aluminum, copper, or different alloys of each.

In another embodiment, the active cooling system includes a heat sink operatively connected to the array of miniature Peltier elements adapted to heat and cool the thermal grill.

In still another embodiment, the heat sink is formed of aluminum, copper, or different alloys of each.

In yet another embodiment, the array of a plurality of miniature Peltier elements are positioned or sandwiched intermediate the thermal grill and the heat sink.

In another embodiment, the medical device includes a disposable sterile patient interface adapted to ensure sterility of the medical device and clinical hygiene during use in clinical environments.

In yet another embodiment, the disposable patient interface includes a thin sheet or film of medical-grade plastic adapted to facilitate effective heat transmission from the medical device to a patient.

In still another embodiment, the disposable patient interface includes a hard plastic rim structured and arranged to facilitate attachment to a bottom portion of the medical device.

In an embodiment, a method of use of the system to apply a quantified, non-damaging pain stimulus to a patient is disclosed.

These and other features, aspects, and advantages of the present invention will become apparent to those skilled in the art from the following detailed description of preferred embodiments taken in connection with the accompanying drawings, which are briefly summarized below, and by reference to the appended claims.

### BRIEF DESCRIPTION OF THE DRAWINGS

Referring now to the attached drawings which form a part of this original disclosure:

FIG. 1A is a top right isometric view of the medical device in accordance with an embodiment of the present invention illustrating features of the housing or casing portion thereof;

FIG. 1B is a bottom right isometric view of the medical device shown in FIG. 1A;

FIG. 2A is a top plan view of the casing containing the elements of the medical device of the present invention;

FIG. 2B is a bottom plan view of the casing shown in FIG. 2A;

FIG. 3A is a front side elevation view of the casing illustrated in FIGS. 2A and 2B;

FIG. 3B is a back or rear side elevation view of the casing illustrated in FIGS. 2A, 2B, and 3A;

FIG. 3C is a right side elevation view of the casing illustrated in FIGS. 2A, 2B, 3A, and 3B;

FIG. 3D is a left side elevation view of the casing illustrated in FIGS. 2A, 2B, 3A, 3B, and 3C;

FIG. 4A is a front side elevation view of the medical device of the present invention illustrating a user’s left hand in position thereon during use of the device;

FIG. 4B is a top right isometric view of the medical device shown in FIG. 4A illustrating a user’s right hand in position thereon during use of the device;

FIG. 4C is a bottom right isometric view of the medical device shown in FIG. 4A;

FIG. 5A is a top right isometric view of the disposable patient interface member in accordance with an embodiment of the present invention;

FIG. 5B is a bottom right isometric view of the disposable patient interface member illustrated in FIG. 5A;

FIG. 6A is a top plan view of the disposable patient interface;

FIG. 6B is a bottom plan view of the disposable patient interface;

FIG. 7A is a front side elevation view of the disposable patient interface;

FIG. 7B is a rear side elevation view of the disposable patient interface;

FIG. 8A is a right side elevation view of the disposable patient interface;

FIG. 8B is a left side elevation view of the disposable patient interface;

FIG. 9 is an exploded top right isometric view of the medical device and the disposable patient interface placed for positioning on the bottom surface thereof;

FIG. 10A is a bottom right isometric view of the medical device shown in FIG. 1B having the disposable patient interface releasably secured to the bottom surface thereof;

FIG. 10B is an exploded bottom right isometric view of the medical device and disposable patient interface illustrated in FIG. 10A;

FIG. 11 is a bottom plan view of the medical device having the disposable patient interface releasably secured to the bottom surface thereof as shown in FIGS. 9, 10A, and 10B;

FIG. 12 is an exploded diagram of the elements of the medical device and the internal electronics assembly or device stack portion thereof illustrated in FIGS. 13A and 13B in accordance with an embodiment of the present invention;

FIG. 13A is a bottom left front isometric view of the internal electronics assembly portion of the medical device of the present invention;

FIG. 13B is a top left rear isometric view of the internal electronics assembly portion of the medical device of the present invention as shown in FIG. 12;

FIG. 14A is a top plan view of the internal electronics assembly portion of the medical device of the present invention illustrated in FIG. 12;

FIG. 14B is a bottom plan view of the internal electronics assembly portion of the medical device of the present invention illustrated in FIGS. 12 and 14A;

FIG. 15A is a front side elevation view of the internal electronics assembly portion of the medical device of the present invention illustrated in FIGS. 12, 14A, and 14B;

FIG. 15B is a rear side elevation view of the internal electronics assembly portion of the medical device of the present invention illustrated in FIGS. 12, 14A, 14B;

FIG. 16A is a right side elevation view of the internal electronics assembly portion of the medical device of the present invention illustrated in FIGS. 12, 14A, 14B, 15A, and 15B;

FIG. 16B is a left side elevation view of the internal electronics assembly portion of the medical device of the present invention illustrated in FIGS. 12, 14A, 14B, 15A, 15B, and 16A;

FIG. 17 is an exploded isometric view of the internal electronics assembly portion of the medical device of the present invention;

FIG. 18 is a flow diagram of a method of general use of the system of the present invention;

FIG. 19 is a flow diagram of a process for using the system of the present invention for assessment of consciousness;

FIG. 20 is a flow diagram of a process for using the system of the present invention in pain research;

FIG. 21 is a flow diagram of a process for using the system of the present invention to evaluate pain disorders in regions of the human body;

FIG. 22 is a flow diagram of a process for using the system of the present invention to assess relative pain tolerance in patients;

FIG. 23 is a flow diagram illustrating the steps of applying multimodal integration features of the system of the present invention;

FIG. 24 is a flow diagram of a method of collecting datasets for longitudinal tracking of patient responses in accordance with the present invention;

FIG. 25 is a flow diagram of a method of analyzing the datasets collected in accordance with the method illustrated in FIG. 24; and

FIG. 26 is a diagram of a data processing network model adapted to receive, store, and process data collected with the system of the present invention.

### DETAILED DESCRIPTION OF THE PREFERRED EMBODIMENTS

Selected embodiments of the present invention will now be explained with reference to the drawings. It will be apparent to those skilled in the art from this disclosure that the following descriptions of the embodiments of the present invention are provided for illustration only and not for the purpose of limiting the invention as defined by the appended claims and their equivalents.

### System Overview

The sensory stimulation system or device of the present invention, referred to hereinafter as the “system” or the “device” 10, comprises a self-contained, portable medical device or single handheld unit designed for one-handed operation at a place of treatment such as a patient’s bedside. As will be described in greater detail below, the system integrates thermal stimulation, precision temperature control and modulation, active cooling, and an ergonomic housing into the single handheld unit. The major subsystems include: a housing or casing that contains all internal components; a patient interface or thermal grill structure; Peltier element arrays that generate a thermal differential to create the thermal grill illusion in the patient; a temperature control system built around a microcontroller with thermistor feedback; an active cooling system with heat sink and fans; a rechargeable battery and power management electronics; a software control system with failsafe protections; and a disposable sterile patient interface for clinical hygiene.

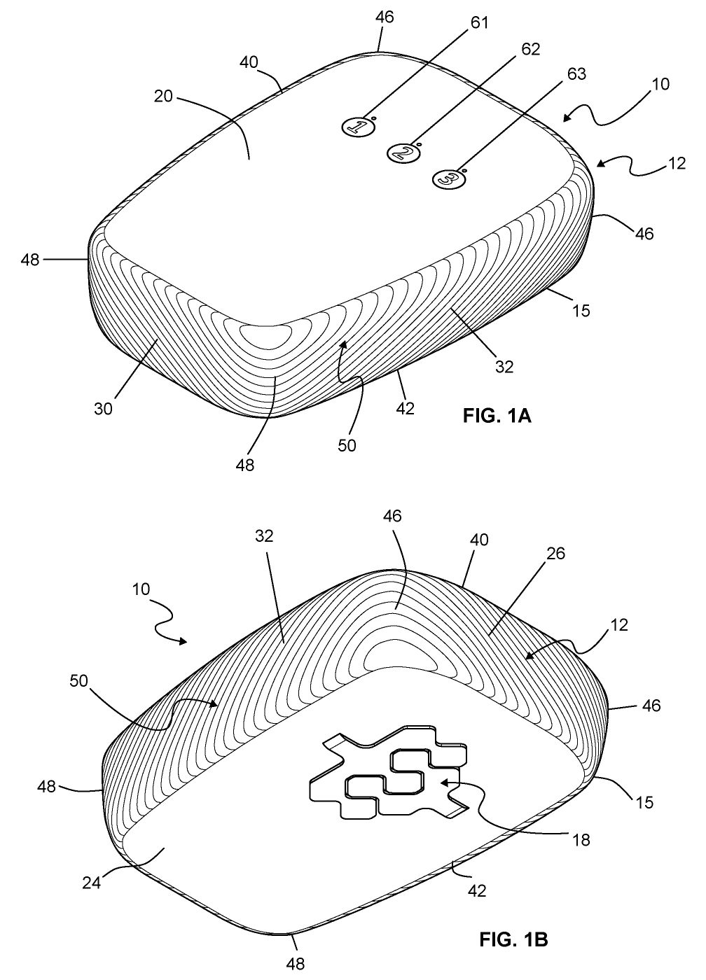

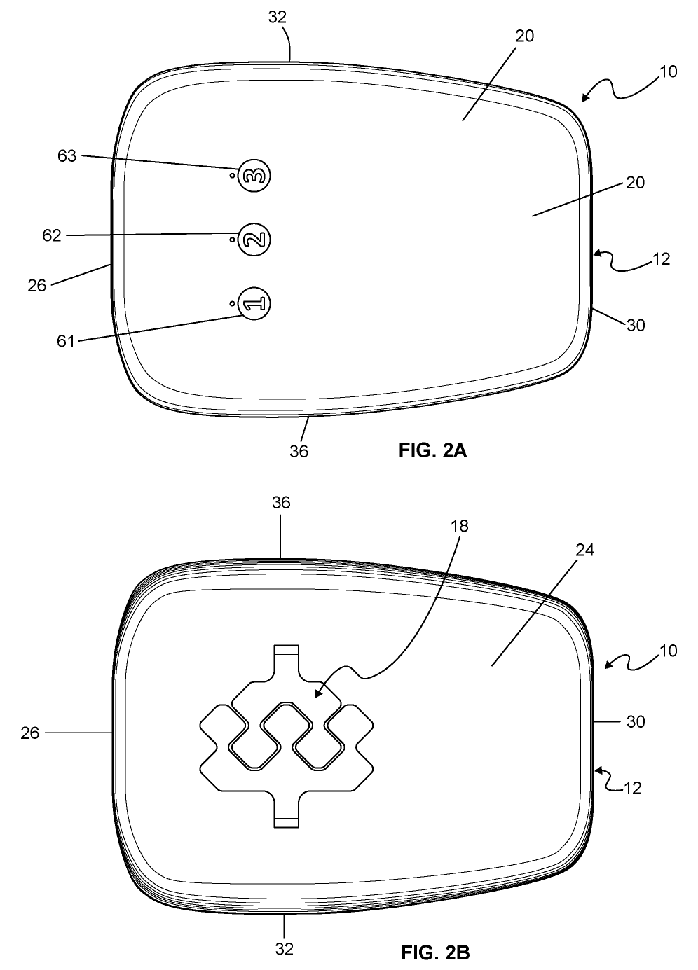

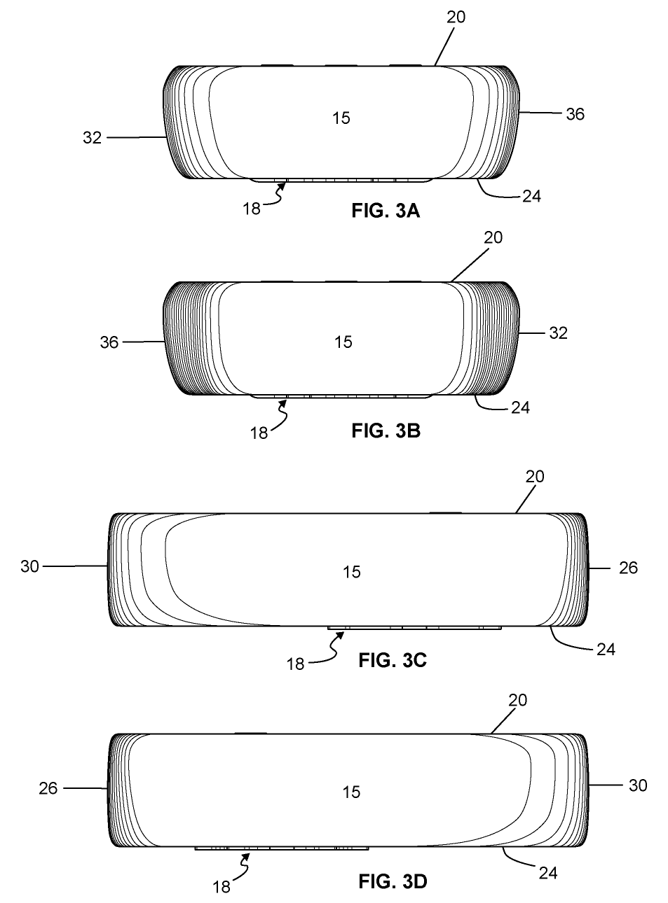

Referring now to FIGS. 1-3, elements of the sensory stimulation system or device 10 of the present invention are shown in greater detail and include a single handheld unit 12 having a hollow housing, casing, or body 15 approximately the size of a smartphone but thicker, thereby providing a compact and portable form factor suitable for one-handed clinical operation. The housing is adapted to contain all of the internal components of the system which are operatively connected to a geometrically unique patient interface structure or shield forming a thermal grill 18. For purposes of simplicity, the patient interface structure or shield will be referred to herein simply as “the thermal grill”. The thermal grill may be either positioned in an aperture or cut out in a lower surface or face 24 of the housing, or, in an embodiment where the housing is of unitary construction and manufactured, for example via plastic injection molding, the thermal grill may be insert-molded with the housing whereby the molded material seamlessly surrounds and secures it. Notwithstanding the foregoing, other materials, manufacturing methods, and form factors may also be used without departing from the scope of the present invention. The housing includes an upper surface 20, the lower surface or face 24 noted above, and front 26, rear 30, right side 32, and left side 36 surfaces, the front, rear, left, and right side surfaces all extending intermediate and peripherally around the upper and lower surfaces, each side surface being operatively connected to the upper and to the lower surfaces by a continuous rounded upper and lower edge 40, 42 respectively. Each of the front, rear, right, and left side surfaces are interconnected by a pair of spaced apart front and rear rounded corners 46, 48 and are further sloped gradually inward as they extend downward from the continuous rounded upper edge 40 to the continuous rounded lower edge thereof, thereby forming a peripherally extending, continuous ergonomic gripping surface 50. The continuous rounded upper and lower edges, the downwardly inwardly sloping side surfaces, and the spaced apart front and rear rounded corners cooperate to configure the shape of the housing which allows the operator to hold and manipulate the entire device comfortably and securely with one hand while applying the stimulus directly to the patient with controlled pressure.

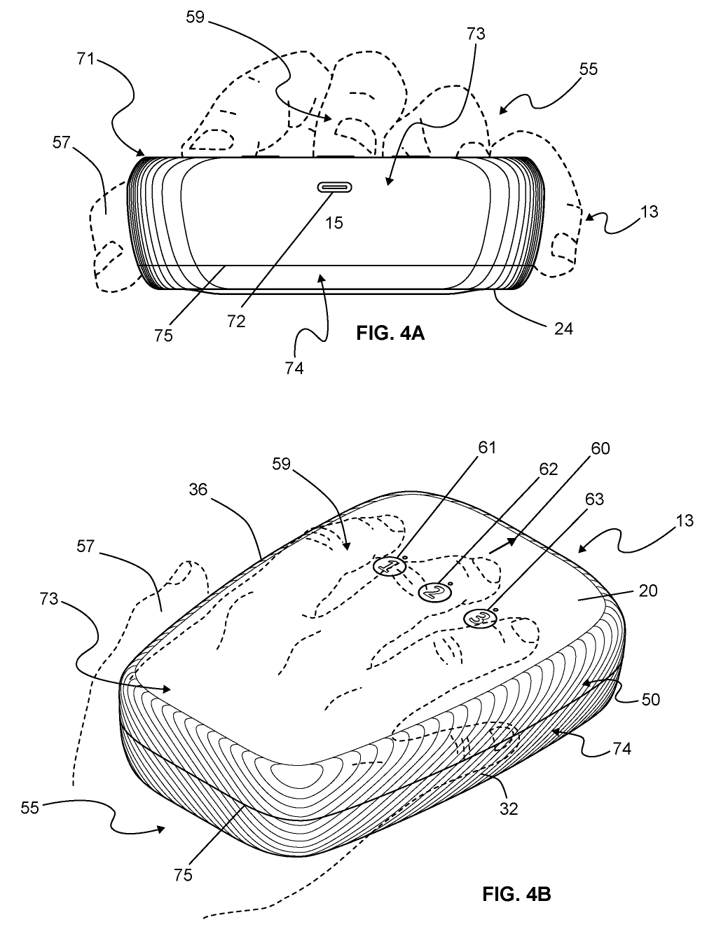

Referring to FIGS. 4A-4C, the embodiment 13 of a handheld unit, like embodiment 12 discussed above, is designed to be held directly by an operator rather than incorporating a separate handle or guide member, the peripherally extending, continuous ergonomic gripping surface 50 serving as the primary grip surface. The inwardly sloping lower edge 42 conforms to the natural curvature of the hand shown in dotted lines at 55, allowing the operator to grip the device between a thumb 57 and several fingers 59 in a pinch-style or palm-style hold. The top surface 20 may include a forward direction indicator 60 to orient the device during application. In the embodiment shown, one or more control actuators or buttons 61, 62, 63 for adjusting stimulus intensity and activating the system are positioned on the top surface 20 for easy access during operation. However, the control actuators may be positioned on the front or side surfaces in other embodiments without departing from the scope hereof. The compact form factor and integrated grip design allow a clinician to retrieve the device from its storage location, power it on, adjust settings, and apply the stimulus to the patient all with a single hand. In embodiment 13, a thin vent slot 70 is formed along a lower portion of one or both of the side surfaces 32, 36 to provide ventilation for the removal of heat from the internal components. The front end includes a charging port 72 for a rechargeable battery which will be described in greater detail below; although, wireless or magnetic charging, for example in the same manner as certain mobile cellphones are charged, may also be used without departing from the scope of the instant invention.

The embodiment 13 illustrated in FIGS. 4A-4C further includes a casing or body 71 having a top body portion 73 and a bottom body portion 74 adapted to be releasably secured to the top body portion along a snap-fit interface 75 extending circumferentially about the casing or body or by snaps, clips, threaded fasteners, or other securement devices suitable for the application. When the top and bottom body portions are secured together, they form an internal cavity or chamber 77 adapted to house all of the thermal stimulation system’s internal components including but not limited to the system’s operating, control and interface systems and to protect them from exposure to potentially damaging contaminants that may be present in an operating environment.

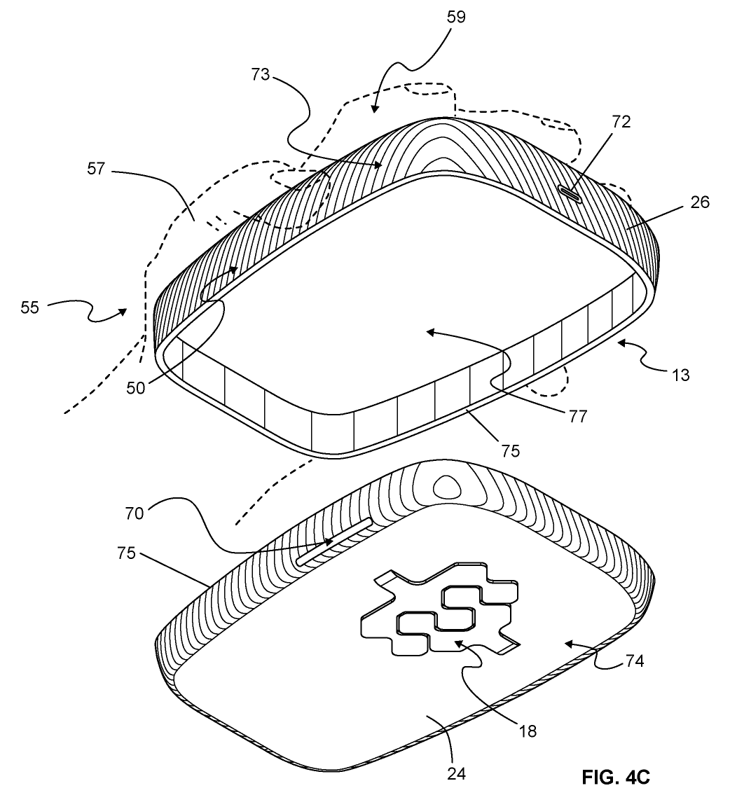

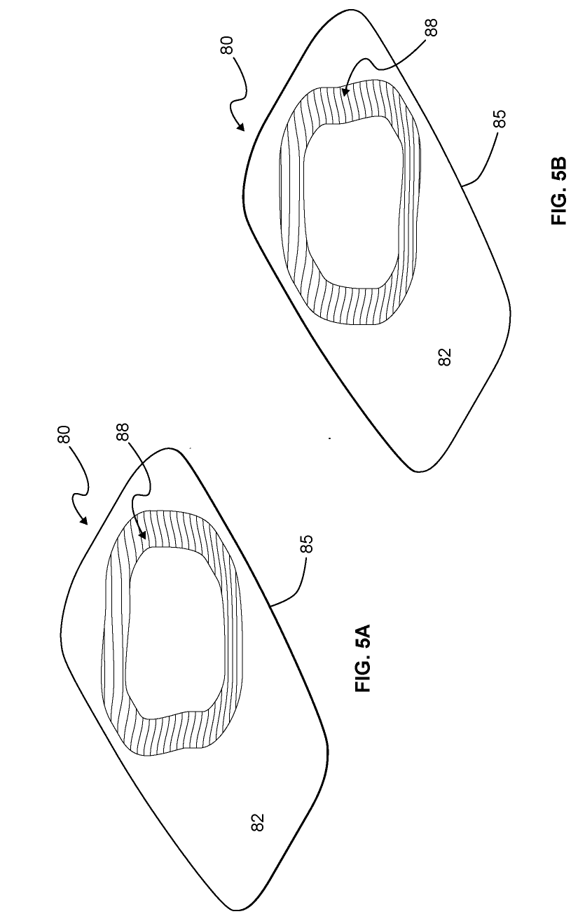

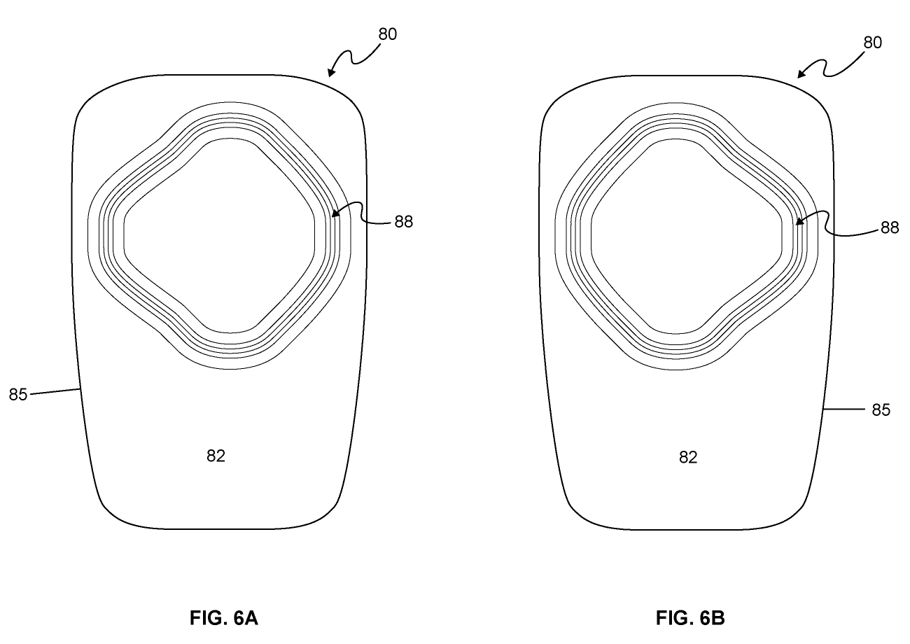

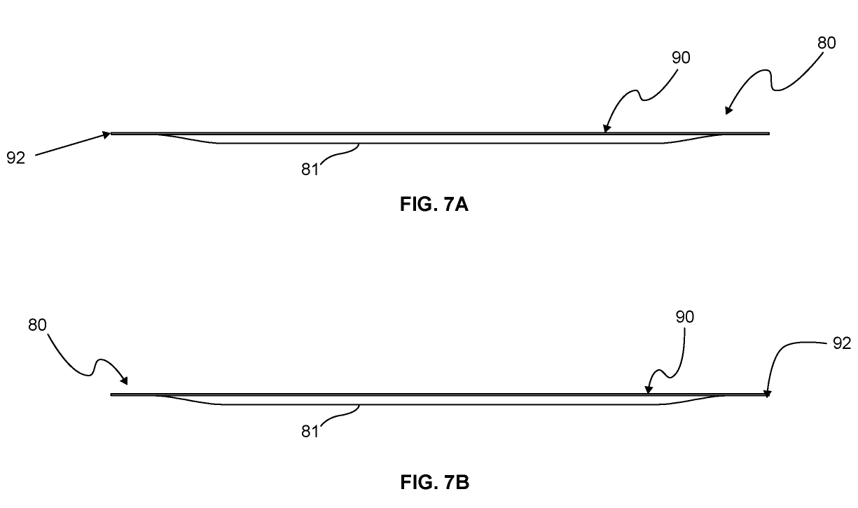

The sensory stimulation system 10 further includes a disposable patient interface 80, the details of which are depicted in FIGS. 5-8. The disposable patient interface is adapted to maintain hygiene and prevent cross-contamination between patients. In the embodiment shown, the disposable patient interface includes a thin film 82 formed of a suitable heat conducting material such as a heat conducting synthetic or plastic, the thin film being supported by a frame or border 85 adapted to maintain the shape of the thin film prior to affixing it in position on the lower surface or face 24 of the handheld unit 12 covering the geometrically unique thermal grill 18 to maintain a sanitary surface that is applied to a patient’s skin. In the embodiment shown, the film includes a plurality of concentric circles 88 adapted to aid in the proper positioning of the film over the top of the thermal grill. The film is sufficiently flexible as to stretch over the thermal grill, thereby forming a bulge or protuberance 81 in the thin film 82 as illustrated in the elevation views thereof in FIGS. 7A, 7B, 8A, and 8B.

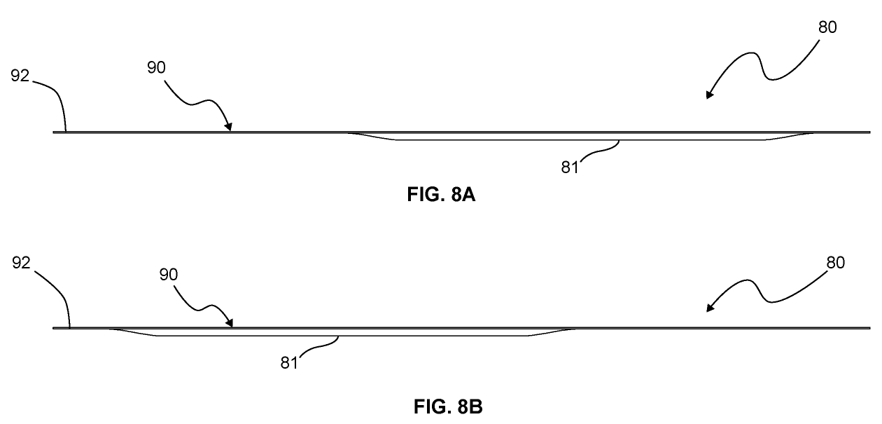

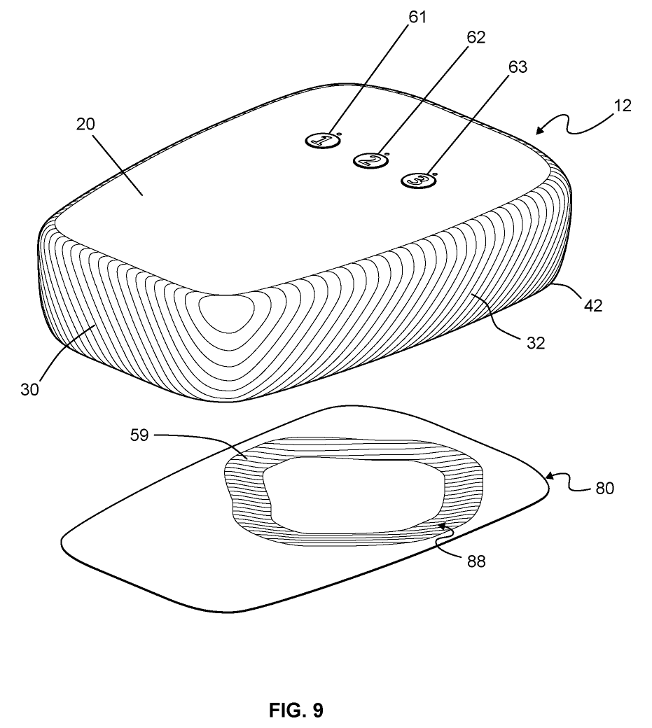

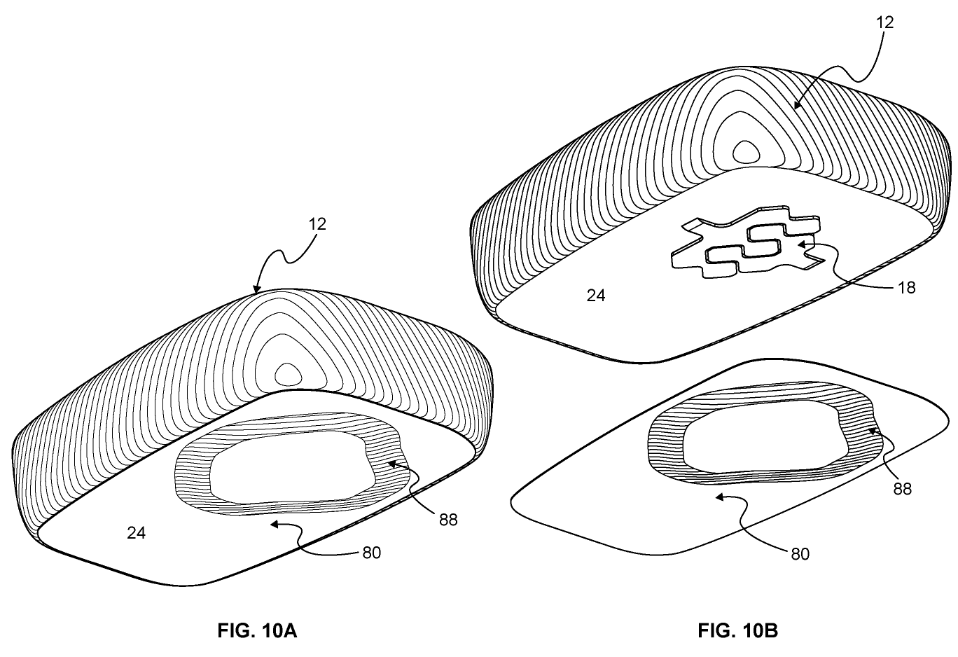

The application and positioning of the disposable patient interface on the lower surface 24 of the handheld unit 12 is best viewed in FIGS. 9-11. The plurality of concentric circles 88 includes a laterally offset or elongated portion 89 adapted to cover the asymmetric configuration of the thermal grill 18. The film and frame or border is releasably secured in position via an adhesive coating 90 applied to a bottom surface 92 of frame or border 85. In other embodiments, the disposable patient interface 80 may be releasably secured in position via one or more clips or other fasteners suitable for the application. The entire assembly is removed and discarded after each patient use.

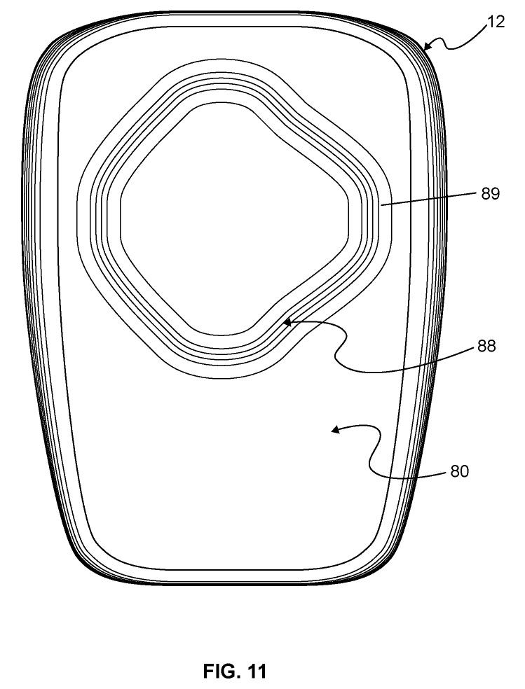

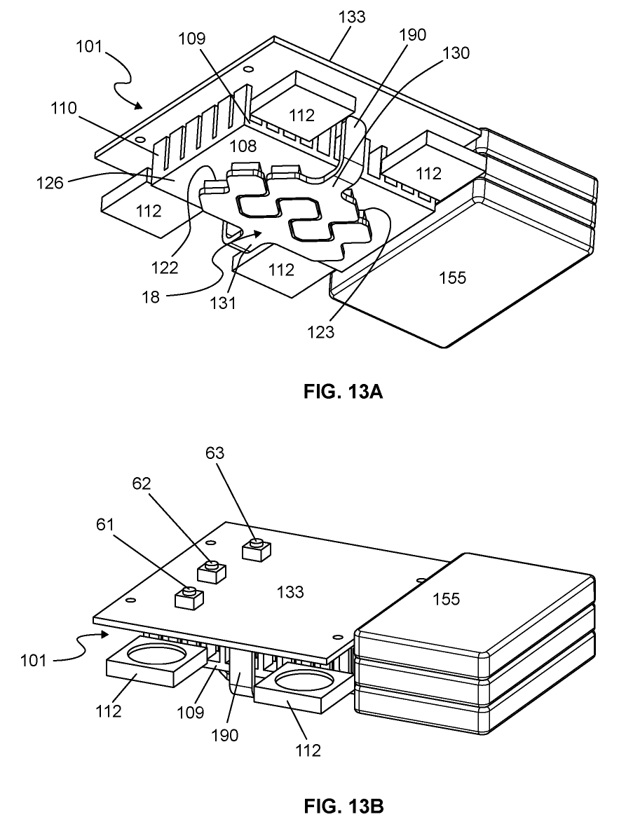

Referring now to FIGS. 12-17, the features of the control elements, the thermal stimulation system, the heat dissipation system and arrangement of the device stack 101 will be discussed in greater detail. Using a patient's skin as an initial reference point, the device layers are arranged in the following order: the patient's skin is in contact with the disposable interface film 82, which covers the patient interface structure or shield which forms the thermal grill 18, as the terms may be used interchangeably herein. At least two uniformly spaced apart patterned Peltier element banks or arrays 100, 104 are positioned intermediate the thermal grill and a heat dissipation device or heat sink 108. The heat sink is preferably of unitary construction, formed of a heat conducting material such as copper, aluminum, or alloys thereof. It includes a flat body member 109 with a lower surface 126 with which the Peltier devices are in operative engagement, and a plurality of uniformly spaced, rectangularly shaped legs 110 operatively connected to an upper surface 127 of the flat body member and extending perpendicularly outward therefrom, thereby forming a heat conducting array 111. A plurality of fans 112 are located in the hollow housing or casing 15. In the embodiment shown, four such fans are used; however, it is to be understood that a lesser or greater number of fans may be employed depending upon the needs of the particular system and the sizes and efficiencies of the fans selected for the application. The fans draw heat through the heat sink legs 110 and the at least two banks or arrays 100, 104, and direct it via the vent slot 70 to the surrounding environment where the heat is dissipated.

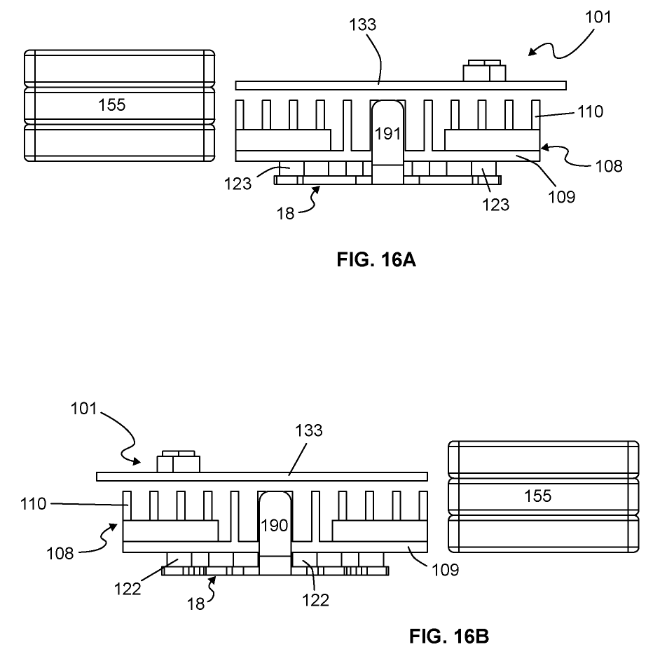

Referring to FIGS. 12 and 17, the arrangement of the Peltier elements is shown in greater detail. Each of the at least two banks or arrays includes a plurality of Peltier elements or modules 120 and 121, each having a top surface 122 and 123, respectively arranged in a respective one of the uniformly spaced apart patterns or arrays 100, 104 with their top surfaces adjacent to and in operative engagement or physical contact with a bottom surface or face 126 of the heat sink 108. The Peltier elements, devices or modules, also referred to in the art as thermoelectric coolers (“TEC”), as the terms are used interchangeably herein, are each constructed of n-type and p-type semiconductor materials having a ceramic plate on opposing sides of the device and which are connected to an external electrical power source. An electrical current passing through a Peltier device generates a heat differential between the interface of the two different materials. This heat differential is known as the Peltier effect. One side of the Peltier element heats up, while the other side cools. The Peltier element arrays are depicted separately in FIG. 12 for purposes of illustration; however, in accordance with the unique structure of the elements of the instant invention, both Peltier element arrays 100 and 104 are configured or arranged in an interlaced relationship in operative engagement with the single heat dissipation device or heat sink 108 as noted above. Each Peltier element further includes a respective bottom surface 128, 129 in contacting engagement with and supported by segments 130 and 131 of the heat shield, respectively. A printed circuit board (PC board) 133 is positioned at the top of the device stack 101 and is secured thereto by suitable fasteners such as screws or clamps, as is known in the art. The one or more control actuators or buttons 61, 62, 63 are operatively connected thereto and adapted to be accessed by a user via apertures formed in the top surface 20 of the handheld device 12.

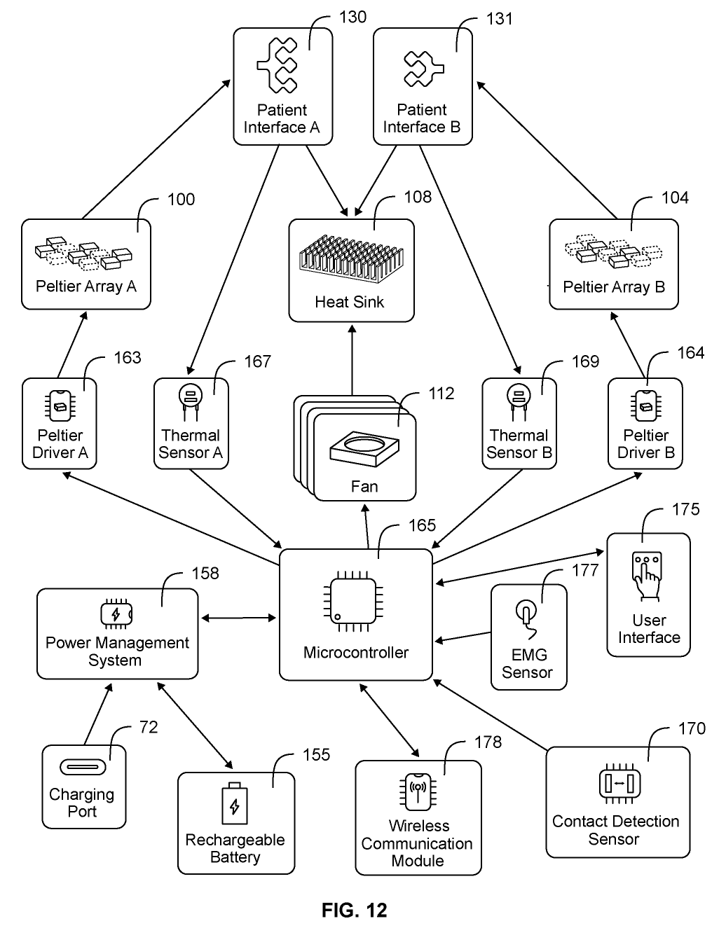

As depicted in greater detail in FIGS. 13B, 14B, and 17, the interlocked relationship of the arrays covered by segments 130 and 131 of the heat shield forms the geometrically unique thermal grill 18. By way of illustration and not of limitation, each of the segments is formed of copper, aluminum, or alloys thereof. However, it is to be understood that they may also be formed of other materials having suitable thermal conductivity properties without departing from the scope of the present invention. The segments 130 and 131 are configured to fit together in locking engagement in a manner analogous to the mating pieces of a jigsaw puzzle. Segment 130 includes at least two fingers 160, 162 extending laterally outward from a body portion 142, the body portion 142 and the at least two fingers lying in a plane. Each finger has a diagonally oriented square end portion 154, 156. The body portion, each finger, and each diagonally oriented end portion are adapted to support a Peltier device 122 positioned thereon and in operative engagement with or adjacent to the lower surface 126 of the heat sink 108. Similarly, segment 131 includes at least three fingers 180, 182, and 184 extending laterally outward from a body portion 144, the body portion 144 and the at least three fingers lying in a plane. Each finger has a diagonally oriented square end portion 186, 187, and 189. The body portion, each finger, and each diagonally oriented end portion are adapted to support a Peltier device 123 positioned in operative engagement with or adjacent to the lower surface 126 of the heat sink 108. The body portions, the fingers and the diagonally oriented end portions of each of the members 130 and 131 are adapted to fit in interlocking engagement with one another in the same plane in a manner analogous to the interlocking mating pieces of a jigsaw puzzle, thereby forming the geometrically unique thermal grill 18, as hereinabove described.

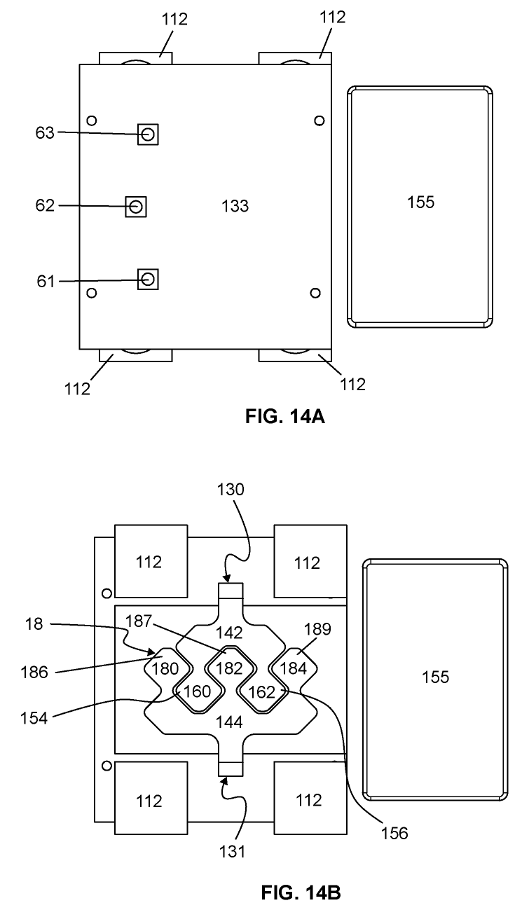

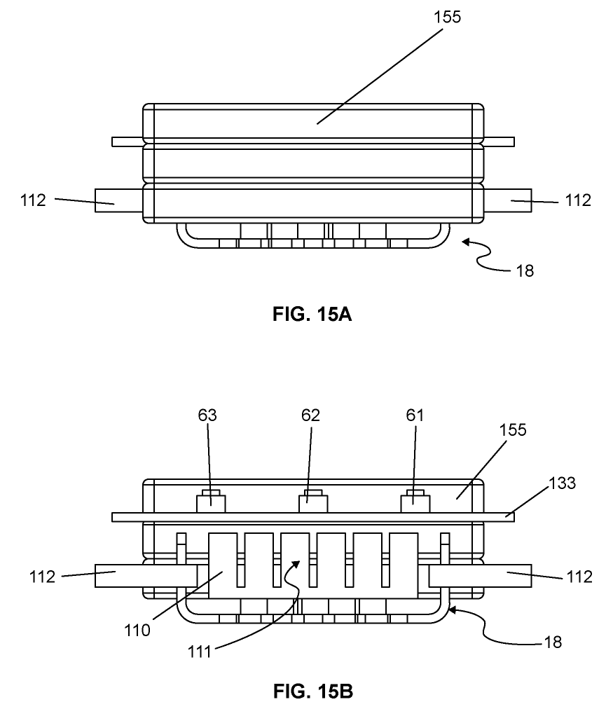

As shown in FIGS. 15A, 15B, 16A, and 16B, when assembled each of first and second segments 130, 131 is adapted to fit over and support a respective one of the banks or sets 100, 104 of Peltier elements or modules 120, 121, respectively, thereby sandwiching the Peltier devices between the lower surface or face 126 of the heat dissipation device or heat sink 108 and a respective upper surfaces 138, 140 of the first and second segments 130, 131 of the geometrically unique thermal grill 18. Each of first and second segments 130, 131 further includes an L-shaped bracket or arm 190, 191, respectively, each L-shaped bracket or arm having a flat portion 192, 193 operatively connected to or integrally formed at a first end thereof 194, 195 with a respective segment 130, 131 and a second end 196, 197 of the flat portion operatively connected to or integrally formed with an upward extending portion 198, 199 thereof. Each of the L-shaped brackets are structured and arranged to cooperate with the heat sink 108 and the housing 12 to maintain the positions of the segments 130, 131 in operative engagement with a respective one of the banks or sets 100, 104 of Peltier elements or modules 120, 121.

Referring to FIG. 17, in operation, bottom surfaces of the Peltier elements in one of the arrays, for example bottom surfaces 128 of the Peltier elements in array 100, will be hot, and bottom surfaces 129 of the other Peltier element array 104 will be cold, or vice versa. The temperature of the bottom surfaces of the Peltier elements in each of the arrays is transferred to an upper surface 138, 140 of each of the segments 130 and 131, respectively and thereafter conducted via a segment’s respective body portion 142, 144 and lower surface 148, 150 thereof to the surface of a patient’s skin. The hot and cold temperatures of the interlaced Peltier element arrays generate a uniform temperature distribution or gradient having a range of approximately 20° C on the cold surfaces to approximately 40° C on the hot surfaces across the hot and cold surfaces of the segments 130 and 131. The uniformly distributed temperature gradient creates the thermal grill illusion described above when applied to the skin simultaneously, namely a sensory phenomenon which creates a disproportionate painful burning sensation, without actually being hot or cold enough to damage the skin.

Referring again to FIG. 12, the system further includes a power source 155, which, in the embodiment shown, is in the form of a rechargeable battery operatively connected to a power management system 158. The power management system 158 is further coupled to charging port 72 to receive electrical power from an external source for recharging the battery 155. The power management system cooperates with Peltier drivers 163, 164 which deliver controlled voltages to the Peltier elements and a microcontroller 165 to modulate the voltage applied to the Peltier devices 120, 121 in each of the banks 100 and 104, respectively. Thermal sensors 167, 169 are operatively connected to each of the first and second segments 130, 131, respectively which form the geometrically unique patient interface structure or thermal grill 18, as hereinabove described. The thermal sensors provide temperature data from each of the segments to the microcontroller 165. Additional heat generated by the Peltier elements is dissipated by the heat sink 108 and fans 112 in response to temperature data provided by the thermal sensors. The microcontroller will allow for users to modulate the intensity of the generated pain stimulus via a user interface 175, which includes the one or more control actuators 61, 62, and 63, by altering the temperature gradient across the hot and cold metal segments 130, 131. The user interface 175 provides a plurality of preselected intensity levels, each corresponding to a respective temperature differential between segments 130, 131 within the device's operating range, with a greater differential producing a more intense thermal grill illusion and a lesser differential producing a milder sensation. The microcontroller will use the thermal sensor-generated temperature data to adjust Peltier drivers 163 and 164 to control the voltage applied to the circuits containing the hot and cold Peltier elements in response to changes in the detected temperature(s). The microcontroller will also use the thermal sensor data to initiate a fail-safe protocol in which the direction of current will be reversed (equalizing the temperature gradient) if a hot side temperature of 45° C or higher is detected.

A contact detection sensor 170 is integrated into the device 12 and is in communication with the microcontroller 165 and a contact detection system. The sensor, by way of example and not of limitation, capacitive in nature, detects when the device interface has made proper contact with a patient's skin. The system would record and communicate contact time and duration data to an external device or clinical system, thereby enabling objective documentation of exactly when and for how long the stimulus was applied. This capability supports clinical recordkeeping, quality assurance, and research applications by providing precise, automated timing data rather than relying on manual clinician documentation.

In an embodiment of the system, an EMG (electromyography) sensor 177 is either integrated into or operatively connected to the device to measure the patient's muscular response to the pain stimulus in real time. The EMG sensor 177 is operatively connected to the microcontroller 165, which is configured to receive and process EMG signals from the sensor. This would provide an objective, quantifiable measurement of patient response, rather than relying solely on subjective clinician observation. An integrated EMG sensor could detect subtle motor responses—microswitches, fasciculations, or graded withdrawal responses—that may be missed by visual observation alone. This addition transforms the device from a stimulus-only tool into a complete stimulus-and-response measurement system.

The system further includes a wireless communication module 178 operatively connected to the microcontroller 165. The wireless communication module 178 is configured to transmit data, including stimulus parameters, contact detection data, EMG sensor data, and operational status information, to one or more external devices, clinical systems, or remote servers via a wireless communication protocol. The wireless communication module 178 may comprise any suitable wireless communication technology, including but not limited to Bluetooth, Bluetooth Low Energy, Wi-Fi, or cellular communication. This capability enables real-time data transmission for clinical recordkeeping, remote monitoring, and integration with electronic health records or other clinical information systems.

Software is used to control the system operation and has two main functions. The first is to modulate the voltage supplied to the circuits containing the hot and cold Peltier elements in order to control the intensity of the pain stimulus consistently, as described above. The second function is to initiate the fail-safe feature noted earlier that equalizes the hot-cold temperature gradient in the situation where a hot side element temperature equal to or exceeding 45° C is detected to prevent any risk of thermal injury to the patient.

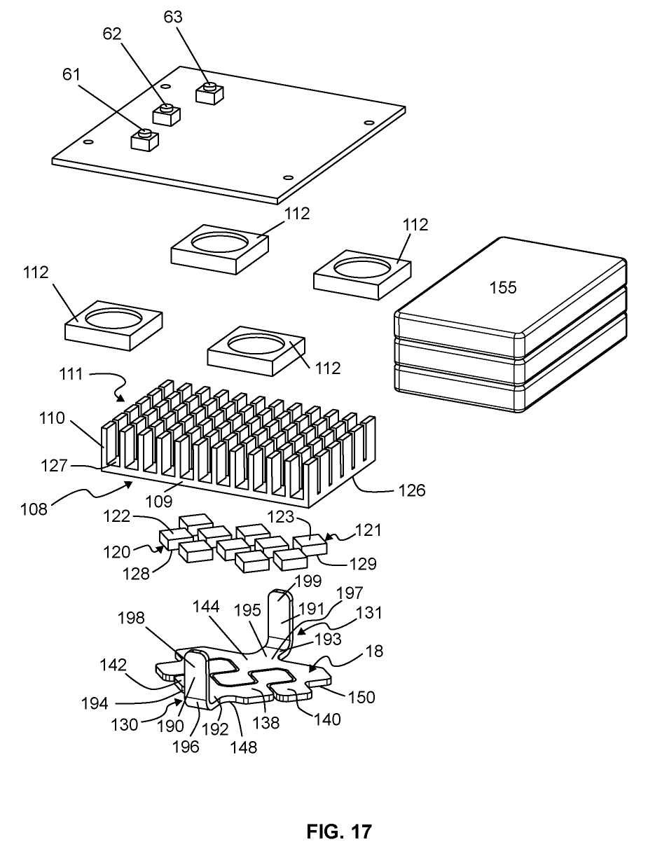

FIG. 18 is a flow diagram of the steps of a method for use of the system in general applications thereof. At step A, a clinician turns on the system 10 and, at step B, adjusts the intensity of the non-harmful pain stimulation to a desired level. At step C, the device heats to a preselected correct temperature for the application. When the preselected correct temperature for the application is reached, at step D the device signals to the clinician that the stimulation treatment may be commenced. At step E, the device interface structure or shield 18 is applied to the non-harmful pain stimulus recipient’s skin and, at step F, the device is pulled away from the recipient’s skin after the non-harmful pain stimulus is felt. At step G, the recipient’s response to the stimulus is assessed. If no recipient response is detected or if the recipient’s response indicates further applications are required, the non-harmful pain stimulation intensity is adjusted if necessary, or, if the stimulus is being applied at a point of patient care, an explicit escalation protocol is applied, and steps B through G are repeated until satisfactory results are observed. After completion of the examination, at step H, the responses are interpreted and recorded, and the device is turned off at step I.

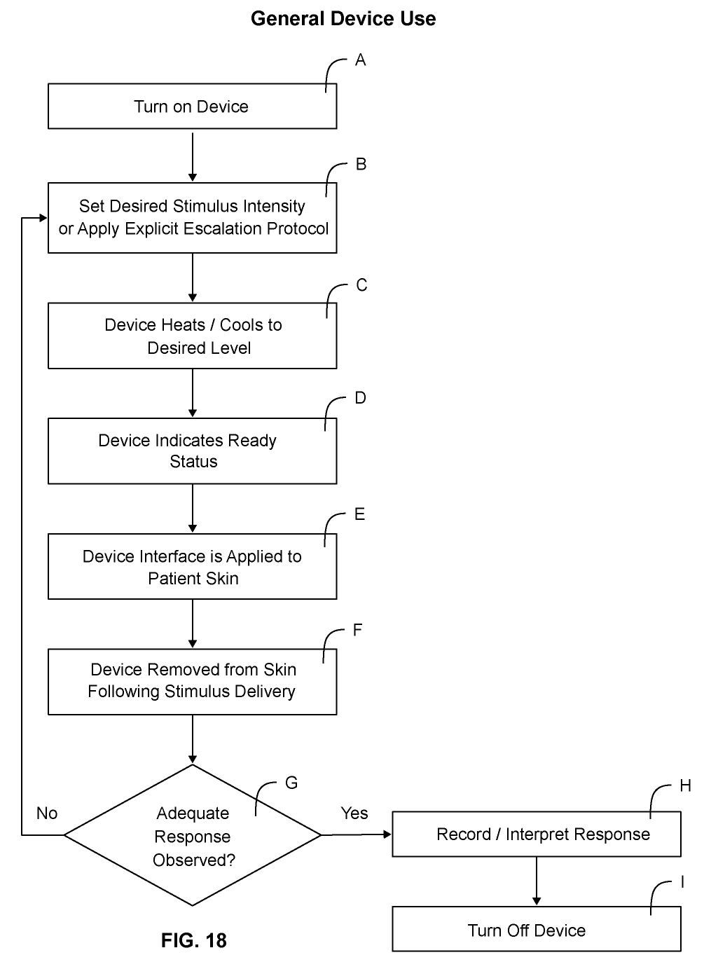

The steps of a method for using the system 10 to assess consciousness are set out in FIG. 19. An important application for the system of the present invention involves intraoperative monitoring for assessing anesthesia effectiveness and depth of sedation. The standardized, quantifiable stimulus enables objective assessment of whether a patient is adequately anesthetized during surgical procedures, potentially improving patient safety and anesthetic management. At step A, the provider prepares the patient for consciousness assessment. At step B, the provider performs the method for general device use set forth in FIG. 18. At step C, the provider assesses the patient’s consciousness based on the patient’s response to the stimulus. At step D, the provider determines whether additional stimulus data is needed; if so, steps B and C are repeated until no further stimulation is required. At step E, the device is turned off and returned to its storage location.

For use in any ICU, operating room, emergency department, or pre-hospital environment, the workflow follows a similar escalation protocol as hereinabove described in the methods set forth in FIGS. 18 and 19. The provider identifies the need to assess a patient's consciousness, retrieves the device from its designated location in the unit, and brings it to the patient's bedside. The provider then uses the device on the patient following the general process described above, beginning at the lowest intensity setting. The provider observes the patient for a response to the painful stimulus. If no response is observed, the provider increases the stimulus intensity on the device and reapplies until a response is detected. If a response is observed, the provider does not increase the intensity further. The provider then notes the intensity level at which the patient responded and the type of response observed. Finally, the device is switched off and placed back in its original storage location. This method applies across all acute care settings and ensures a consistent, standardized assessment regardless of the clinical environment. Moreover, the system and its methods of application are designed for use across a broad range of clinical environments. In the Neuro-ICU, the system serves as a primary tool for assessing consciousness and monitoring neurological responsiveness in patients with disorders of consciousness. In general ICU settings, it supports sedation monitoring and routine neurological evaluation. In the emergency department, the system enables rapid neurological examination as part of acute patient assessment. In the operating room, it provides intraoperative monitoring capabilities, and in pre-hospital care settings, it allows field neurological assessment by paramedics and emergency medical personnel.

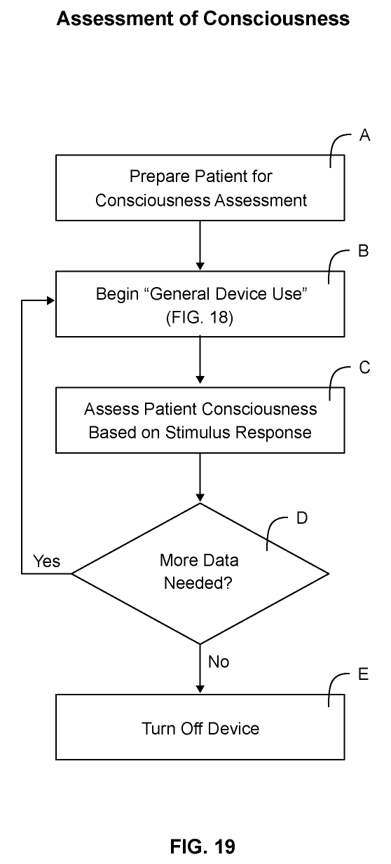

The steps of a method for using the system 10 to conduct pain research are set out in FIG. 20. At step A, the investigator establishes the stimulus parameter conditions to be investigated. At step B, the investigator performs the method for general device use set forth in FIG. 18 for each of the individual parameter conditions established in step A. At step C, the investigator records the subjects’ responses to each stimulus parameter selection. At step D, the investigator determines if additional parameter conditions and/or subjects require testing. If the conclusion in step D is affirmative, steps B through D are repeated until the investigator determines that no further testing is required. At step E, the data collected from all parameter conditions and subjects tested is aggregated. At step F, the aggregated data from step E is analyzed and reported.

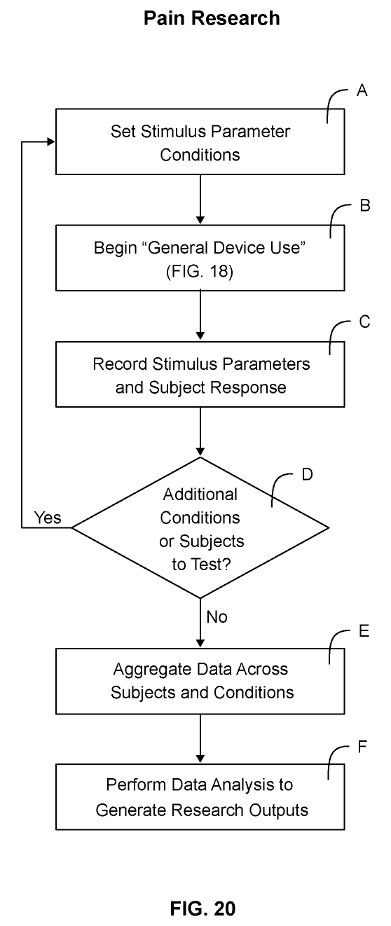

Referring now to FIG. 21, a method of evaluating pain disorders in different regions of the human body is disclosed. The device can be applied to assess regional pain conditions by comparing patient responses across different body areas. Because the stimulus is perfectly standardized, any variation in patient response between body regions reflects genuine differences in sensory function rather than differences in how the stimulus was applied. This capability supports diagnosis and monitoring of conditions such as complex regional pain syndrome (CRPS), peripheral neuropathies, and other regional sensory disorders. The steps of the method begin at step A, where the specific regions of the body are determined for evaluating a specific pain disorder. At step B, the points of application of the pain stimulus in the specific regions identified in step A are determined. At step C, the investigator performs the method for general device use set forth in FIG. 18 at each of the individual body regions selected in step B. At step D, the results collected at step C are evaluated to determine if testing of additional body regions is required. If the determination is affirmative, steps B through D are repeated until it is determined that testing of additional regions is not required. At step E, the stimulus response thresholds across the different body regions assessed identify significant variations in response thresholds. At step F, the presence and/or severity of regional pain disorders are quantified.

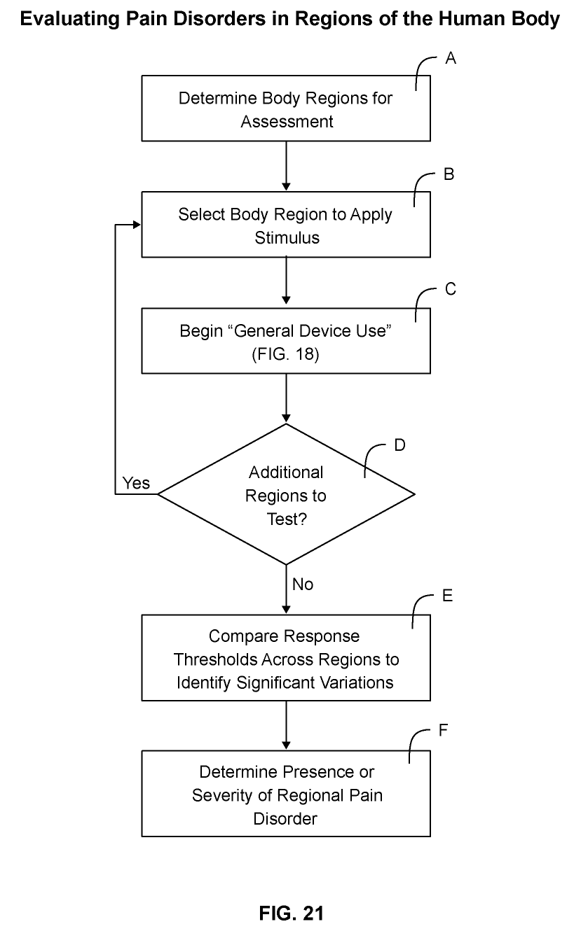

FIG. 22 lays out the steps for a method of measuring relative pain tolerance. The system's calibrated intensity levels can be used to quantify an individual patient's pain threshold and tolerance. By systematically increasing stimulus intensity and recording the level at which a response is observed, clinicians can establish a quantitative pain sensitivity profile. Because this device provides one of the first truly standardized pain stimuli, measuring many patients' responses at different intensity levels could enable the creation of a population-level pain tolerance distribution. A physician prescribing a pain management medication, or needing to understand a patient's relative pain sensitivity for any clinical reason, could use the device to assess the patient and place them within this distribution based on their response profile. This has applications in pre-surgical planning, chronic pain management, personalized analgesic dosing, and baseline assessment for patients beginning pain-related therapies. The steps of the method begin at step A, the collection of patient demographic data. At step B, the investigator performs the method for general device use set forth in FIG. 18. A patient’s response to pain stimulus is recorded at step C, and at step D, the patient’s response is compared to a demographic-based reference scale. At step E, the patient’s relative pain tolerance is determined (i.e., low, average, high).

A particularly powerful application involves longitudinal tracking of pain responsiveness combined with integration across multiple physiological monitoring modalities. The system can be paired with pupillometry to correlate pain response with pupillary dilation patterns, providing an additional objective marker of neurological function. Heart rate variability (HRV) monitoring can assess the autonomic nervous system's response to the stimulus, adding cardiovascular data to the assessment. Combined with the EMG integration described above, this creates a multimodal neurological assessment platform that fuses thermal grill stimulus delivery with simultaneous physiological monitoring across multiple sensor types. Longitudinal data tracking across these modalities enables trend analysis and clinical decision support dashboards that provide a comprehensive picture of a patient's neurological trajectory over time. This application is tightly coupled with the hereinabove described data integration system, which provides the infrastructure for collecting, synchronizing, and analyzing data across all of these modalities.

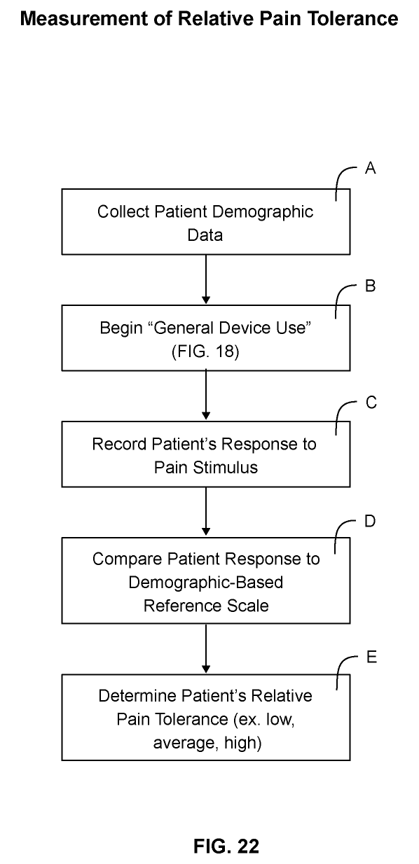

FIG. 23 illustrates the steps of multimodal integration which begins at step A, selecting the external monitoring systems to be used. At step B, the system is operatively connected to and synchronized with the external monitoring systems selected in step A. At step C, the investigator elects to either perform the method for general device use set forth in FIG. 18, step D, or to collect physiological data from external systems, step E. At step F, the investigator records time-synchronized stimulus parameters and physiological responses, and at step G, the investigator stores the integrated multimodal dataset.

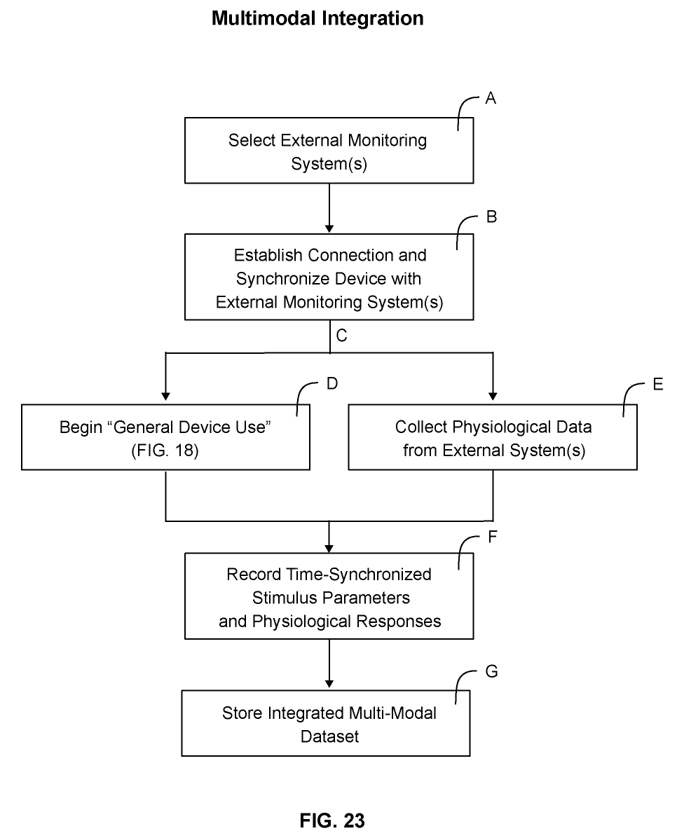

FIG. 24 lays out the steps of a method for longitudinal tracking, a particularly powerful application which involves longitudinal tracking of pain responsiveness combined with integration across multiple physiological monitoring modalities. The system can be paired with pupillometry to correlate pain response with pupillary dilation patterns, providing an additional objective marker of neurological function. Heart rate variability (HRV) monitoring can assess the autonomic nervous system's response to the stimulus, adding cardiovascular data to the assessment. Combined with the EMG integration described above, this process creates a multimodal neurological assessment platform that fuses thermal grill stimulus delivery with simultaneous physiological monitoring across multiple sensor types. Longitudinal data tracking across these modalities enables trend analysis and clinical decision support dashboards that provide a comprehensive picture of a patient's neurological trajectory over time. This application is tightly coupled with the data integration system described herein, which provides the infrastructure for collecting, synchronizing, and analyzing data across all of these modalities. The process of collecting a dataset begins at step A, performing the steps of the general device use set forth in FIG. 18. At step B, the investigator records stimulus parameters and responses via manual entry or automatic device logging, which is stored at step C in a local or connected data system. At step D the investigator queries to determine if any additional testing is required. If the response is affirmative, the investigator elects to either perform the method for general device use set forth in FIG. 18 until it is determined that no further testing is required, whereupon the process is terminated at step E.

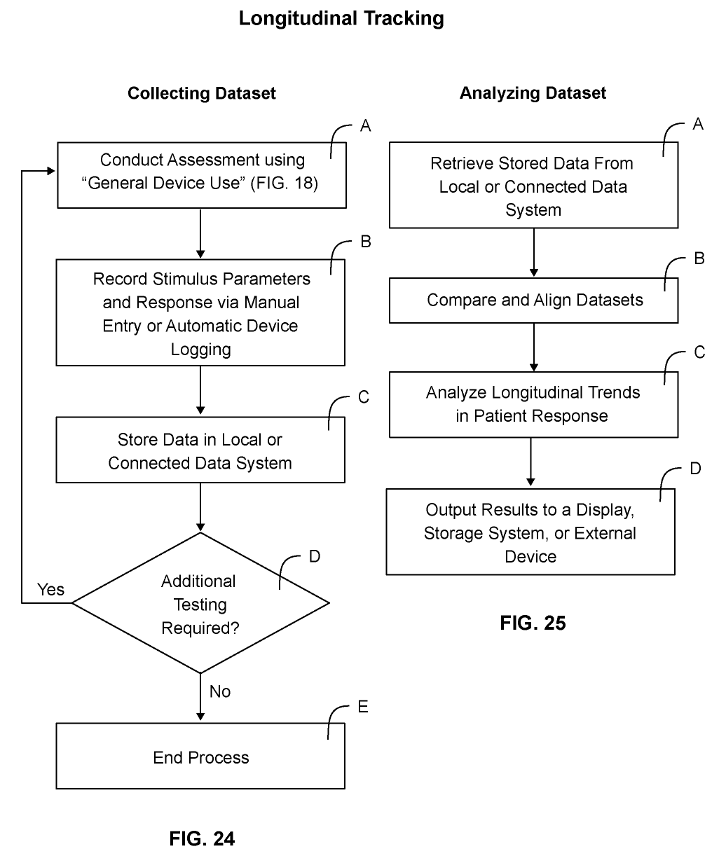

FIG. 25 lays out the steps of a method of analyzing the contents of a dataset. At step A, an investigator retrieves stored data or datasets from a local or from a connected data system. The data or datasets are compared and aligned at step B, and next, at step C, any identified longitudinal trends in patient response are analyzed. At step D, the results of the analysis performed in step C are sent in the form of output to a display, to a storage system or to an external device.

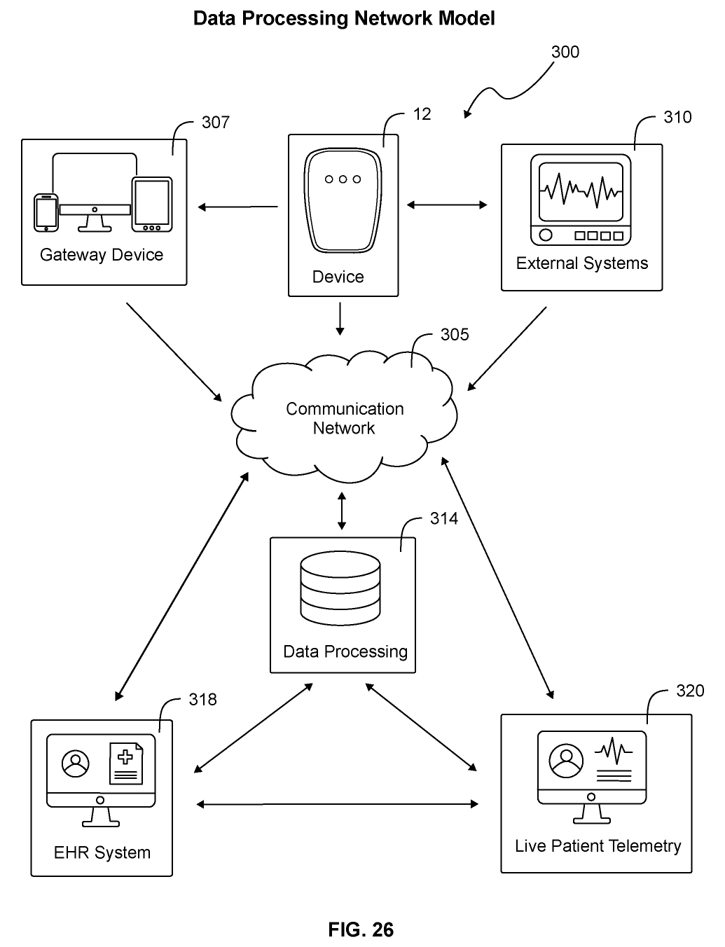

The system of the present invention provides a comprehensive data integration system and methods for tracking and utilizing examination results. The central concept is providing a unified tracking system alongside the device that captures all stimulus and response data in one place. This data could be entered directly on the device itself, through a companion phone application that pairs with the device via Bluetooth or similar wireless protocol, or through another connected clinical device. The system includes persistent data storage capability for recording the results of each pain stimulus test, including stimulus parameters, contact timing from the contact detection system shown in FIG. 12, and patient response data whether observed by the clinician or measured by integrated sensors. Because the contact detection system provides precise timing of when the stimulus is applied, the system can correlate the exact moment of stimulus delivery with physiologic responses measured by external monitoring devices such as pupilometers, heart rate variability monitors, and EMG (electromyography) sensors, thereby creating a synchronized, multi-source clinical dataset. The system interfaces with Electronic Medical Records (EMRs) for automatic documentation of examination results, thus eliminating manual charting. Integration with live charting systems used in ICU and NCCU (neurocritical care unit) settings allows real-time data flow into the patient's clinical record. Beyond documentation, the system incorporates algorithm-based clinical recommendations derived from accumulated patient data, supporting longitudinal tracking of responsiveness and trend analysis for neurological trajectory assessment. Accordingly, a data processing network model 300 is provided to facilitate execution of the above-described functions as shown in FIG. 26. In the system, the handheld device 12 is in communication either via Bluetooth, wireless or wire connection to data network or cloud storage system 305, a gateway device 307 and/or external systems 310 such as pupilometers, EKG devices, and the like. The data network, in turn is in selective or simultaneous communication with a data processing center or function 314, one or more electronic health records (EHR) systems 318, and to patients via a live patient telemetry system 320. The possibilities are endless.

While only selected embodiments have been chosen to illustrate the present invention, it will be apparent to those skilled in the art from this disclosure that various changes and modifications can be made herein without departing from the scope of the invention as defined in the appended claims. Furthermore, the foregoing descriptions of the embodiments according to the present invention are provided for illustration only, and not for the purpose of limiting the invention as defined by the appended claims and their equivalents.

What is claimed is:

1. A self-contained, portable, handheld, non-invasive thermal stimulation system for assessing a patient’s response to pain stimuli, the system comprising:
a user interface;
a contact detection sensor;
a power supply;
a temperature control system;
a cooling system;
a patient interface structure;
at least two Peltier element banks or arrays positioned intermediate portions of the cooling system and the patient interface structure, each bank or array including a plurality of uniformly spaced apart, patterned Peltier elements;
a disposable patient interface; and
a housing or casing adapted to contain the thermal stimulation system’s internal components.

2. The self-contained, portable, handheld, non-invasive thermal stimulation system of claim 1 wherein the housing or casing includes an upper surface, a lower surface or face, a front surface, a rear surface, a right side surface, and a left side surface, the front, rear, left, and right side surfaces, all extending intermediate and peripherally around the upper and lower surfaces, each side surface being operatively connected to the upper and to the lower surfaces by a continuous rounded upper edge and a continuous rounded lower edge, respectively; the front, rear, right, and left side surfaces further being interconnected by a respective pair of spaced apart front and rear rounded corners.

3. The self-contained, portable, handheld, non-invasive thermal stimulation system of claim 2 wherein each of the front, rear, right, and left side surfaces are sloped gradually inward as they extend downward from the continuous rounded upper edge to the continuous rounded lower edge thereof, thereby forming a peripherally extending, continuous ergonomic gripping surface, the continuous ergonomic gripping surface being adapted to allow an operator to hold and manipulate the housing or casing comfortably and securely with one hand while applying the stimulus directly to the patient with controlled pressure.

4. The self-contained, portable, handheld, non-invasive thermal stimulation system of claim 3 wherein the user interface comprises one or more control actuators operatively connected to the temperature control system.

5. The self-contained, portable, handheld, non-invasive thermal stimulation system of claim 4 wherein the power supply is a rechargeable battery.

6. The self-contained, portable, handheld, non-invasive thermal stimulation system of claim 5 wherein the temperature control system comprises a Peltier driver operatively connected to each of the plurality of uniformly spaced apart, patterned Peltier elements forming the at least two Peltier element banks or arrays, each Peltier driver being adapted to apply a controlled voltage to each of the plurality of uniformly spaced apart, patterned Peltier elements to which it is operatively connected, a thermal sensor operatively connected to each of the at least two Peltier element banks or arrays, a microcontroller operatively connected to each of the Peltier drivers, the thermal sensors, the user interface, and the cooling system; the microcontroller being adapted to adjust the respective Peltier drivers to control or modulate the applied voltage to the Peltier bank or array to which each is connected in response to user inputs via the user interface and to Peltier element bank or array temperature feedback data from each of the thermal sensors.

7. The self-contained, portable, handheld, non-invasive thermal stimulation system of claim 6 wherein the cooling system comprises:
a heat sink including a flat body member having a lower surface and an upper surface, the lower surface being in operative contact or engagement with each of the at least two Peltier banks or arrays, the heat sink further including a plurality of uniformly spaced, rectangularly shaped legs operatively connected to the upper surface of the flat body member and extending perpendicularly outward therefrom, thereby forming a heat conducting array; and
a plurality of fans adapted to cooperate with the heat sink to dissipate heat generated by each of the plurality of uniformly spaced apart, patterned Peltier elements of the at least two Peltier element banks or arrays.

8. The self-contained, portable, handheld, non-invasive thermal stimulation system of claim 7 wherein the housing or casing adapted to contain all of the thermal stimulation system’s internal components further includes a vent slot formed along a lower portion of one or both of the right and left side surfaces, the one or both vent slots being adapted to receive heat drawn by each of the plurality of fans through the heat sink legs and to direct the heat to the surrounding environment whereby the heat is dissipated.

9. The self-contained, portable, handheld, non-invasive thermal stimulation system of claim 8 wherein the patient interface structure comprises a first segment and a second segment, the first segment having a body portion, at least two fingers extending outward from the body portion, each finger including a diagonally oriented end portion, the body portion, the at least two fingers and diagonally oriented end portions lying in a plane and being adapted to support a first one of the at least two Peltier element banks or arrays, the second segment including at least three fingers extending laterally outward from a body portion, each finger including a diagonally oriented end portion, the body portion and the at least three fingers and diagonally oriented end portions lying in a plane and being adapted to support a second one of the at least two Peltier element banks or arrays, the body portions, the fingers and the diagonally oriented end portions of each of the first and second segments being further adapted to fit in interlocking mating engagement with one another, thereby forming a geometrically unique thermal grill.

10. The self-contained, portable, handheld, non-invasive thermal stimulation system of claim 9 wherein each of the first and second segments further includes an L-shaped bracket, each bracket having a first flat portion operatively connected to or integrally formed at a first end thereof with a respective one of the segments and an upward extending portion at a second end, each of the L-shaped brackets being adapted to cooperate with the heat sink and the housing to maintain the positions of the first and second segments in operative engagement with a respective one of the banks or arrays of Peltier elements.

11. The self-contained, portable, handheld, non-invasive thermal stimulation system of claim 10 wherein the disposable patient interface is releasably secured to the lower surface of the housing or casing and adapted to cover the thermal grill.

12. The self-contained, portable, handheld, non-invasive thermal stimulation system of claim 11 wherein the disposable patient interface includes a thin film supported by a frame or border adapted to maintain the shape of the thin film prior to affixing it in position on the lower surface of the housing or casing.

13. The self-contained, portable, handheld, non-invasive thermal stimulation system of claim 12 wherein the disposable patient interface includes an adhesive coating applied to a bottom surface of the frame or border, the adhesive coating being adapted to releasably secure the disposable patient interface in position on the lower surface of the housing or casing.

14. The self-contained, portable, handheld, non-invasive thermal stimulation system of claim 6 further including a wireless communication module operatively connected to the microcontroller.

15. The self-contained, portable, handheld, non-invasive thermal stimulation system of claim 6 further including an electromyography (EMG) sensor operatively connected to the microcontroller.

16. A method for using a self-contained, portable, handheld, non-invasive thermal stimulation system having a handheld stimulation device for assessing a patient’s response to pain stimuli, the method comprising the steps of:
a. turning on the system;
b. either adjusting the intensity of pain stimulation to a desired level or applying an explicit stimulus escalation protocol;
c. heating the handheld stimulation device to a preselected temperature suitable for the application;
d. waiting for the handheld stimulation device to signal that the preselected temperature has been reached and that the stimulation treatment may be commenced;
f. removing the handheld stimulation device from the recipient’s skin after the non-harmful pain stimulus is felt;
g. assessing the patient’s response to the stimulus;
h. adjusting the stimulus intensity if no response is detected or if further applications are required;
i. repeating steps b. through h. until satisfactory results are observed;
j. interpreting and recording the responses; and
k. turning off the system.

17. A method for using a self-contained, portable, handheld, non-invasive thermal stimulation system having a handheld stimulation device for assessing a patient’s consciousness, the method comprising the steps of:
a. preparing the patient for consciousness assessment;
b. turning on the system;
c. either adjusting the intensity of pain stimulation to a desired level or applying an explicit stimulus escalation protocol;
d. heating the handheld stimulation device to a preselected temperature suitable for the application;
e. waiting for the handheld stimulation device to signal that the preselected temperature has been reached and that the stimulation treatment may be commenced;
f. applying the handheld stimulation device to the patient’s skin;
g. removing the handheld stimulation device from the recipient’s skin after the non-harmful pain stimulus is felt;
h. assessing the patient’s response to the stimulus;
i. adjusting the stimulus intensity if no response is detected or if further applications are required;
j. repeating steps c. through i. until satisfactory results are observed;
k. interpreting and recording the responses; and
l. turning off the system.

18. A method for using a self-contained, portable, handheld, non-invasive thermal stimulation system having a handheld stimulation device for performing pain research, the method comprising the steps of:
a. establishing the stimulus parameter conditions to be investigated;
b. turning on the system;
c. either adjusting the intensity of pain stimulation to a desired level or applying an explicit stimulus escalation protocol;
d. heating the handheld stimulation device to a preselected temperature suitable for the application;
e. waiting for the handheld stimulation device to signal that the preselected temperature has been reached and that the stimulation treatment may be commenced;
f. applying the handheld stimulation device to a subject’s skin;
g. removing the handheld stimulation device from the recipient’s skin after the non-harmful pain stimulus is felt;
h. assessing the patient’s response to the stimulus;
i. adjusting the stimulus intensity if no response is detected or if further applications are required;
j. repeating steps c. through i. until satisfactory results are observed;
k. aggregating the data collected from all parameter conditions;
l. analyzing and reporting the collected data; and
m. turning off the system.

19. A method for using a self-contained, portable, handheld, non-invasive thermal stimulation system having a handheld stimulation device for evaluating pain disorders in different regions of the human body, the method comprising the steps of:
a. identifying specific regions of the body for evaluating a specific pain disorder;
b. determining points of application of the pain stimulus in the specific regions identified in step a.;
c. turning on the system;
d. either adjusting the intensity of pain stimulation to a desired level or applying an explicit stimulus escalation protocol for each of the specific regions identified in step a;
e. heating the handheld stimulation device to a preselected temperature suitable for the application;
f. waiting for the handheld stimulation device to signal that the preselected temperature has been reached and that the stimulation treatment may be commenced;
g. applying the handheld stimulation device to the patient’s skin at each of the specific regions identified in step a;
h. removing the handheld stimulation device from the recipient’s skin after the non-harmful pain stimulus is felt;
i. assessing the patient’s response to the stimulus;
j. adjusting the stimulus intensity if no response is detected or if further applications are required;
k. repeating steps d. through j. until satisfactory results are observed;
l. assessing the responses to identify significant variations in response thresholds;
m. quantifying the presence and/or severity of regional pain disorders; and
n. turning off the system.

20. A method for using a self-contained, portable, handheld, non-invasive thermal stimulation system having a handheld stimulation device for measuring relative pain tolerance, the method comprising the steps of:
a. collecting patient demographic data;
b. turning on the system;
c. either adjusting the intensity of pain stimulation to a desired level or applying an explicit stimulus escalation protocol;
d. heating the handheld stimulation device to a preselected temperature suitable for the application;
e. waiting for the handheld stimulation device to signal that the preselected temperature has been reached and that the stimulation treatment may be commenced;
f. applying the handheld stimulation device to the patient’s skin;
g. removing the handheld stimulation device from the recipient’s skin after the non-harmful pain stimulus is felt;
h. assessing the patient’s response to the stimulus;
i. adjusting the stimulus intensity if no response is detected or if further applications are required;
j. repeating steps c. through i. until satisfactory results are observed;
k. recording the patient’s response;
l. comparing the patient’s response to a demographic-based reference scale;
m. determining the patient’s relative pain tolerance;
n. turning off the system.

21. A method for using a self-contained, portable, handheld, non-invasive thermal stimulation system having a handheld stimulation device for tracking pain responsiveness with integration across multiple physiological monitoring modalities, the method comprising the steps of:
a. selecting the external monitoring systems to be used;
b. turning on the system;
c. operatively connecting the system to and synchronizing it with the external monitoring systems selected in step a.;
d. either adjusting the intensity of pain stimulation to a desired level or applying an explicit stimulus escalation protocol;
e. heating the handheld stimulation device to a preselected temperature suitable for the application;
f. waiting for the handheld stimulation device to signal that the preselected temperature has been reached and that the stimulation treatment may be commenced;
h. removing the handheld stimulation device from the recipient’s skin after the non-harmful pain stimulus is felt;
i. assessing the patient’s response to the stimulus;
j. adjusting the stimulus intensity if no response is detected or if further applications are required;
k. repeating steps c. through h. until satisfactory results are observed;
l. interpreting and recording the responses; and
m. turning off the system; or
n. collecting physiological data from external systems;
o. recording time-synchronized stimulus parameters and physiological responses; and
p. storing the integrated multimodal dataset.

22. A method for using a self-contained, portable, handheld, non-invasive thermal stimulation system having a handheld stimulation device for longitudinal tracking of pain responsiveness combined with integration across multiple physiological monitoring modalities, the method comprising the steps of:
a. turning on the system;
b. either adjusting the intensity of pain stimulation to a desired level or applying an explicit stimulus escalation protocol;
c. heating the handheld stimulation device to a preselected temperature suitable for the application;
d. waiting for the handheld stimulation device to signal that the preselected temperature has been reached and that the stimulation treatment may be commenced;
e. applying the handheld stimulation device to the patient’s skin;
f. removing the handheld stimulation device from the recipient’s skin after the non-harmful pain stimulus is felt;
g. assessing the patient’s response to the stimulus;
h. adjusting the stimulus intensity if no response is detected or if further applications are required;
i. repeating steps b. through h. until satisfactory results are observed;
j. recording the stimulus parameters and responses via manual entry or automatic device logging;
k. storing the stimulus parameters and responses in a local or connected data system;
l. querying to determine if any additional testing is required;
m. repeating steps b. through l. if required; and
n. turning off the system.

23. A method for using a self-contained, portable, handheld, non-invasive thermal stimulation system having a handheld stimulation device for analyzing the contents of a dataset, the method comprising the steps of:
a. retrieving stored data or datasets from a local or from a connected data system;
b. comparing and aligning the data or datasets;
c. identifying and analyzing longitudinal trends in patient responses; and
d. sending results of analysis in the form of output to a display, storage system, or external device.
